(19) United States
(12) Patent Application Publication
(10) Pub. No.: US 2004/0089305 A1
(43) Pub. Date: May 13, 2004

(54) Endotracheal tube safety device connector

(76) Inventors: John-Eric S. Vallarta, East Windsor, NJ (US); Peter A. Rienzo, Colts Neck, NJ (US)

Correspondence Address: Charles I. Brodsky, Esq., 2 Bucks Lane, Marlboro, NJ, 07746, US

(21) Appl. No.: 10/291,948

(22) Filed: Nov. 13, 2002

(51) Int. Cl. A61M 16/00

(52) U.S. Cl. 128/207.14

(57) ABSTRACT

A connector including a flange integrally joining input and output conduits together, with the flange including a slot of litmus paper filler of a characteristic to change color when detecting the presence of carbon dioxide deflected into the slot in capturing airflow through the input and output conduits.

### CROSS-REFERENCE TO RELATED APPLICATIONS

NONE

### STATEMENT REGARDING FEDERALLY SPONSORED RESEARCH OR DEVELOPMENT

Research and development of this invention and Application have not been federally sponsored, and no rights are given under any Federal program.

### REFERENCE TO A MICROFICHE APPENDIX

NOT APPLICABLE

### BACKGROUND OF THE INVENTION

1. Field of the Invention

This invention relates to endotracheal tube intubation, in general, and to a safety device connector usable in automatically and instantly detecting for carbon dioxide expired by a patient.

2. Description of the Related Art

As is well known and understood, paramedics and emergency medical technicians are frequently called upon to intubate a person who has stopped, or is experiencing difficulty breathing. In common usage, one end of an endotracheal tube is inserted to maintain or establish an airway, and an opposite end is coupled to a carbon dioxide detector which is in turn coupled to a ventilator or AMBU squeeze bag. Oftentimes, the medical personnel carry such a tube in one pocket, with the detector in a separate pocket, ready for use if need be.

A litmus type paper composition is typically employed within the airflow line of the detector to indicate that the endotracheal tube is properly inserted above the esophagus by a color change produced in response to carbon dioxide being expired by the person. In use, one is required to join the carbon dioxide detector to the endotracheal tube before the optimal monitoring of airflow can take place. As will be readily appreciated, the use of the endotracheal tube occurs during emergency-type situations—where time is of the essence, and in knowing as quickly as possible that air is being delivered to the trachea and whether the patient is breathing or not. A commonly employed carbon dioxide detector is about the size of a cigarette pack, and sells for about $20.00.

As will become clear from the following description, the endotracheal tube safety device of the invention effectively melds the end connector of the endotracheal tube with the previously utilized carbon dioxide detector, in enabling a faster intubation to be had and an almost instantaneous indication of carbon dioxide expiration when present—both at a far less cost.

### SUMMARY OF THE INVENTION

As will be seen from the description which follows, the safety device connector of the invention includes a substantially cylindrical input conduit, a substantially cylindrical output conduit, and a flange circumscribing the two conduits in joining them together. An opening preferably in the form of a slot—is provided internal of the flange in communication with airflow through the conduits once they are joined together. A first means—as with a litmus paper composition—is included within the opening and of a characteristic to provide a visual change in response to a predetermined presence of component particles within the conduits—such as in the nature of carbon dioxide. In accordance with the invention, a second means extends inwardly from an input conduit side of the flange to deflect the component particles toward the visual changing first means.

In a preferred embodiment of the invention, the input conduit is of a larger diameter than that of the output conduit, with their longitudinal axes being respectively co-linear. The component particle deflecting means in this preferred construction is of an orientation to extend perpendicularly to the longitudinal axis of the input conduit in one version, or to extend at an angle with respect to the flange rearwardly toward the output conduit in a second version. With the smaller diameter endotracheal tube coupled to the output conduit, and with the normally larger diameter tube coupling the input conduit to the ventilator, AMBU bag, etc. as in usual utilizations, the safety connector device of the invention will be seen to combine the features of the coupling and the color change monitoring together, just by widening very slightly the flange which previously joined the two conduits together, so as to allow the inclusion within the flange of the slot enclosing the litmus paper composition or like color change indicator.

As will thus be appreciated, the safety device connector of the invention essentially modifies the existing endotracheal tube connector to incorporate within it, the previously separate carbon dioxide detector. Such modified connector could be made available as a separately purchasable device for use with endotracheal tubes employed in this manner—or could be combined with the endotracheal tube as an integral unit. Either version will be seen to eliminate the weight and bulk of the previously separate carbon dioxide detector, and at a significant cost savings.

### BRIEF DESCRIPTION OF THE DRAWINGS

These and other features of the present invention will be more clearly understood from a consideration of the following description, taken in conjunction with the accompanying drawings, in which:

FIG. 1 is an isometric view of a commonly employed endotracheal tube connector helpful in an understanding of the present invention;

FIG. 2 is a cross-sectional view of the connector of FIG. 1 as modified in accordance with one construction of the present invention;

FIG. 3 is a cross-sectional view of the endotracheal tube connector of FIG. 1 as modified in accordance with a second construction of the invention; and

FIG. 4 is a side view of the modified connector according to the teachings of the present invention.

### DETAILED DESCRIPTION OF THE INVENTION

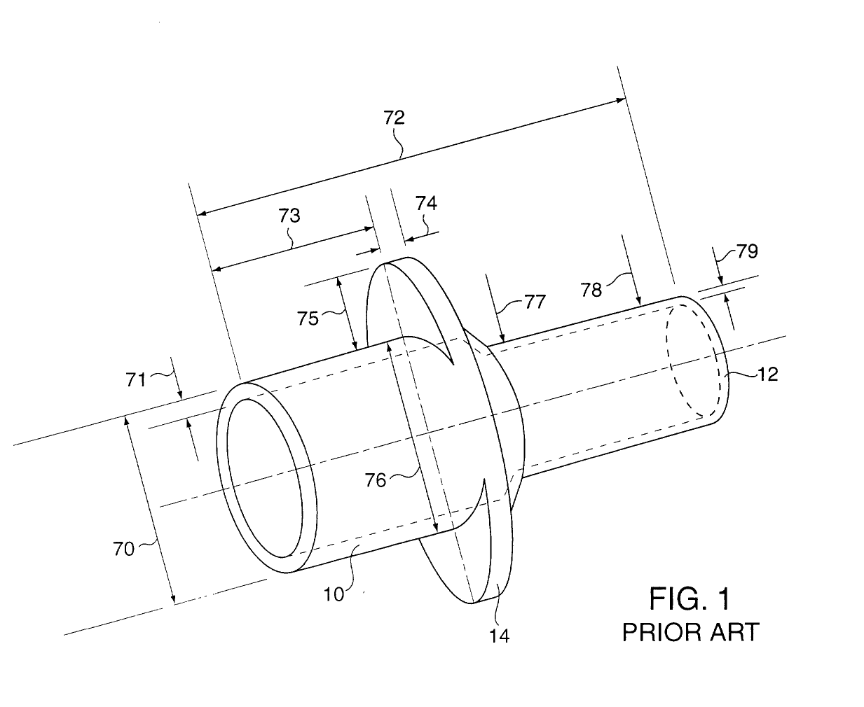

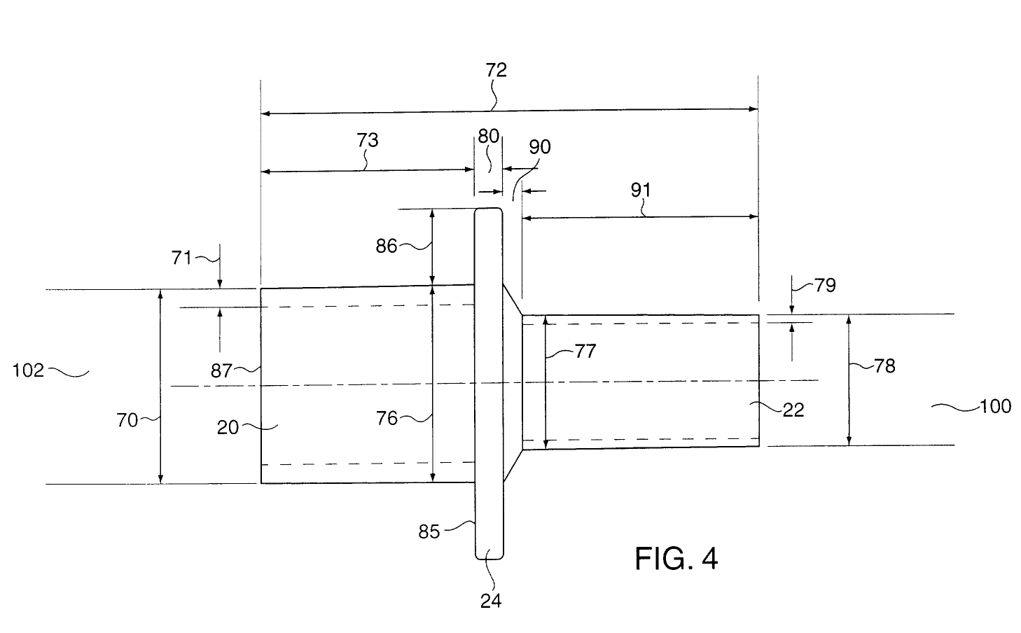

Referring to a typical prior art connector of FIG. 1, reference numeral 10 indicates a substantially cylindrical input conduit for coupling through a carbon dioxide detector to a source of oxygen to be provided to a person in need. Reference numeral 12 indicates a substantially cylindrical output conduit for coupling with an endotracheal tube to intubate such person. A flange 14 circumscribes the input and output conduits 10, 12 in joining the conduits together. The carbon dioxide detector commonly employed couples to the input conduit, and usually employs a litmus paper type composition to change color in response to the presence of carbon dioxide in an airflow expired by a patient in use. The output conduit 12, in turn, couples to the endotracheal tube. As will be understood by those skilled in the art, the connector of FIG. 1 is inserted into one end of the endotracheal tube, shown as 100 in FIG. 4. The coupling to the carbon dioxide detector is shown at 102 in that same drawing.

Various dimensions for the prior art connector of FIG. 1 are as follows:

Reference numeral 70 . . . 0.60 inches
Reference numeral 71 . . . 0.06 inches
Reference numeral 72 . . . 1.55 inches
Reference numeral 73 . . . 0.65 inches
Reference numeral 74 . . . 0.08 inches
Reference numeral 75 . . . 0.20 inches
Reference numeral 76 . . . 0.62 inches
Reference numeral 77 . . . 0.41 inches
Reference numeral 78 . . . 0.38 inches
Reference numeral 79 . . . 0.02 inches

In accordance with the invention, the endotracheal tube connector of FIG. 1 is modified so as to incorporate within it the previously separate carbon dioxide detector. In so doing, and as will be seen below, most of the dimensions of the prior art connector are maintained, so as to cooperate in the exact same manner with the endotracheal tube intubating the patient itself, and in the tubing which couples to the ventilator or AMBU bag. As will also become clear, the modification of the endotracheal tube connector primarily is with the dimensions selected for the circumscribing flange which joins the input and output conduits, and in the internal construction of the flange itself.

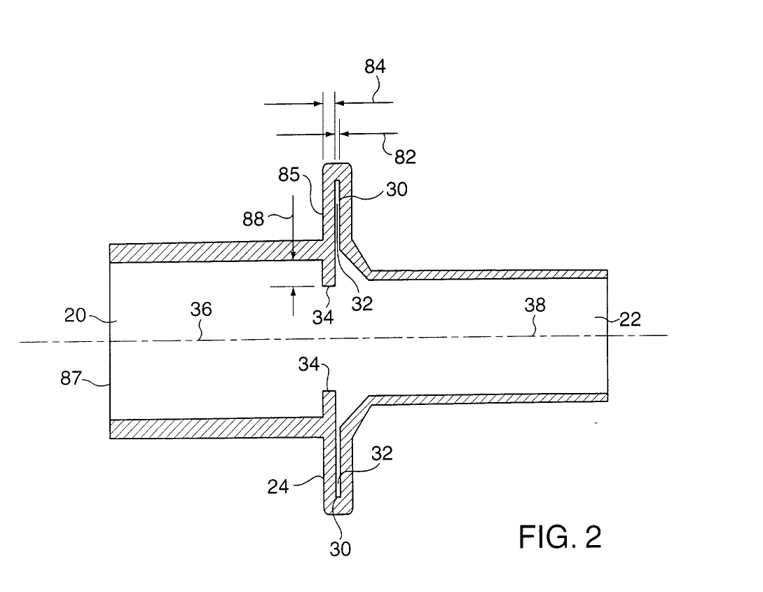

Thus, referring to FIGS. 2, 3 and 4, the substantially cylindrical input conduit of the improved connector is shown at 20 with the same dimensions 70, 71 and 76 as in FIG. 1. The substantially cylindrical output conduit is shown at 22 with the same dimensions 77, 78 and 79. The overall length for the modified connector remains the same, as at 72, as is the dimension 73, locating near the edge surface 85 of the flange (24 in FIGS. 2-4 and 14 in FIG. 1) measured with respect to the left edge surface 87 of the input conduit (20, or 10).

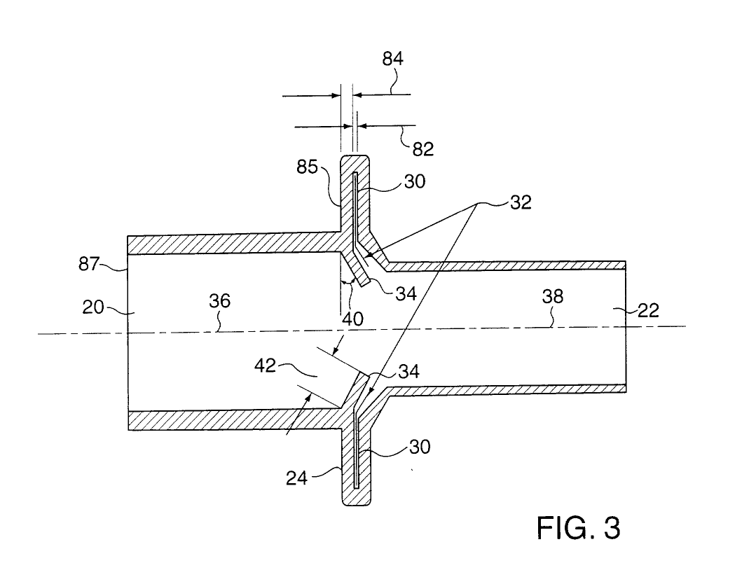

In accordance with the present invention, the flange 24 of FIGS. 2-4 is modified by first providing an opening internal of the flange so as to communicate with airflow through the two conduits 20, 22. Shown, for example by the slot 30 in FIGS. 2 and 3, such opening includes a litmus paper type composition 32 or similar material of a characteristic to produce a visual change in response to the presence of carbon dioxide within the conduits 20, 22. With such visual change preferably being in the nature of a change in color upon detection of carbon dioxide, the endotracheal tube connector of FIGS. 2-4 further includes a wall or like surface 34 which not only defines the opening 30, but serves to extend inwardly into the airflow to deflect carbon dioxide expired from the patient towards the visual change indicator. In the embodiment of FIG. 2, for example, such wall 34 extends perpendicularly to the longitudinal axis 36 of the input conduit 20 while in the embodiment of FIG. 3, the wall 34 extends at an angle with respect to the flange, in a direction rearwardly towards the output conduit 22. Such angle, shown as 40 in FIG. 3, may be of the order of 20°, and with the length 42 of the inwardly extending wall 34 being some 0.1 inch in either case.

With the litmus paper composition material 32 shown fitted within the slot opening 30, and with the longitudinal axis 36 of the input conduit 20 being co-linear with the longitudinal axis 38 of the output conduit 22 (as shown in FIGS. 2-4), modifying the flange 24 in these drawings compared to the flange 14 of the prior art FIG. 1 construction entails the widening of the prior art flange from its 0.08 inch dimension of reference numeral 74 to 0.1 inch, for example, shown as 80 in FIG. 4. With the width 82 of the slot opening 30 shown in FIGS. 2 and 3 as 0.02 inch, for example, the litmus paper type composition can easily be inserted within the opening 30, centered from the edges of the flange on each side by 0.04 inch (reference numeral 84). To accommodate the insertion of the carbon dioxide detector into the opening 30, the flange dimension 75 of the prior art construction (0.20 inch) is likewise slightly increased, to 0.23 inches, shown as 86 in FIG. 4. Where the deflecting airflow wall 34 is perpendicular to the longitudinal axis 36 (FIG. 2) instead of at an angle as in FIG. 3, its length may be reduced somewhat, if desired, to 0.06 inch, for example (reference numeral 88), compared to 0.1 inch (reference numeral 42).

Reference numerals 90 and 91 in these modified constructions may be 0.06 and 0.74 inches, respectively. As will be appreciated, in use, carbon dioxide expired by the patient is deflected by the wall 34 towards the slot opening 30, where its interaction with the litmus paper type composition, or similar material, effects the color change, or like visual alternation. The bulk and weight of the previously employed carbon dioxide detector can thus easily be eliminated, and its significant cost eliminated as well, simply by modifying the flange to incorporate the visual change indicator described above. In such manner, the safety device connector of FIGS. 2-4 could be made available as a separate purchasable unit, or can be simply fitted within the endotracheal tubing itself, and sold as an integral unit, in much the same manner as with the availability and sale of presently available endotracheal tube devices.

While there have been described what are considered to be preferred embodiments of the present invention, it will be readily appreciated by those skilled in the art that modifications can be made without departing from the scope of the teachings herein. Thus, whereas the invention has been described in the context of providing a visual change in response to the presence of carbon dioxide within the conduits of the connector, the improvements of the invention will follow equally as well where the detection is to be instead only in response to a predetermined presence of component particles of any type within the airflow through the conduits in question. Again, whether the flange deflector wall extends perpendicularly to the direction of the airflow, or at an angle with respect to it, those component particles, once present at a predetermined level, will continue to provide the visual change advantageous in detecting their presence within the input conduit. For at least such reason, therefore, resort should be had to the claims appended hereto for a true understanding of the scope of the invention.

We claim:

1. An endotracheal tube safety device connector comprising:
a substantially cylindrical input conduit for coupling with a source of oxygen to be provided to a person in need thereof;
a substantially cylindrical output conduit for coupling with an endotracheal tube to intubate such person;
a flange circumscribing said input and output conduits in joining said conduits together;
an opening internal of said flange in communication with airflow through said conduits when joined together;
first means within said opening of a characteristic to provide a visual change therein in response to the presence of carbon dioxide within said conduits;
and second means extending inwardly into said airflow from an input conduit side of said flange for deflecting carbon dioxide expired from such person into said output conduit towards said first means.

2. The safety device connector of claim 1 wherein said first means is of a litmus paper composition.

3. The safety device connector of claim 2 wherein said litmus paper composition exhibits a change in color upon detection of carbon dioxide.

4. The safety device connector of claim 2 wherein said litmus paper composition fits within a slot in said opening.

5. The safety device connector claim 1 wherein said input conduit is of a diameter greater than the diameter of said output conduit.

6. The safety device connector of claim 1 wherein said second means extends perpendicularly to a longitudinal axis of said input conduit.

7. The safety device connector of claim 1 wherein said second means extends at an angle with respect to said flange rearwardly toward said output conduit.

8. The safety device connector of claim 7 wherein said second means extends rearwardly at an angle of 20 with respect to said flange.

9. The safety device connector of claim 6 wherein said longitudinal axis of said input conduit is co-linear with a longitudinal axis of said output conduit.

10. A safety device connector comprising:
a substantially cylindrical input conduit;
a substantially cylindrical output conduit;
a flange circumscribing said input and output conduits in joining said conduits together;
a slot internal of said flange in communication with airflow through said conduits when joined together;
first means within said slot of a characteristic to provide a visual change therein in response to a predetermined presence of component particles within said conduits; and
second means extending inwardly from an input conduit side of said flange for deflecting said component particles toward said first means.

11. The safety device connector of claim 9 wherein said second means extends perpendicularly to a longitudinal axis of said input conduit.

12. The safety device connector of claim 9 wherein said second means extends at an angle with respect to said flange rearwardly toward said output conduit.

13. The safety device connector of claim 9, in combination with a first tube coupled to said input conduit, and a second tube coupled to said output conduit.
